(19) United States
(12) Patent Application Publication
(10) Pub. No.: US 2007/0191264 A1
(43) Pub. Date: Aug. 16, 2007

(54) Methods for inhibiting the growth of bacteria

(75) Inventors: Robert J. McMahon, Evansville, IN (US); Thomas Cleary, Houston, TX (US); Theresa Ochoa, Lima (PE)

Correspondence Address: BRISTOL-MYERS SQUIBB COMPANY - MEAD JOHNSON, 2400 WEST LLOYD EXPRESSWAY, PATENT DEPARTMENT, EVANSVILLE, IN, 47721, US

(73) Assignee: Bristol-Myers Squibb Company, a Delaware Corporation

(21) Appl. No.: 11/250,677

(22) Filed: Oct. 14, 2005

Related U.S. Application Data
(60) Provisional application No. 60/677,969, filed on May 5, 2005.

(51) Int. Cl. A61K 38/40

(52) U.S. Cl. 514/6

(57) ABSTRACT

The present invention is directed to a novel method for inhibiting the growth of bacterial pathogens expressing a type III secretory system as well as enteroaggregative E. coli and/or preventing or treating an infection caused by the same. The method comprises administering to the subject an effective amount of bovine lactoferrin.

This application claims the priority benefit of U.S. Provisional Application 60/677,969 filed May 5, 2005 which is incorporated by reference herein in its entirety.

### BACKGROUND OF THE INVENTION

(1) Field of the Invention

The present invention relates generally to methods for inhibiting the growth of bacteria.

(2) Description of the Related Art

The gastrointestinal (GI) tract of an infant is colonized by bacteria within hours after birth. The bacterium can be ingested through foods or water or can be obtained directly from individuals handling the infant. One bacterial family that often resides within the human GI tract is Enterobacteriaceae, which consists of faculatively anaerobic Gram-negative rods. A number of genera within this family can be considered human intestinal pathogens, including Escherichia, Salmonella, Shigella, and Yersinia.

Escherichia coli (E. coli) is one of the main species of bacteria that resides in the lower intestine of warm-blooded animals. As part of the normal flora of the human intestinal tract, E. coli plays a crucial role in food digestion by producing vitamin K from undigested material in the large intestine. Once established, an E. coli strain may persist in the GI tract for months or even years. As long as these bacteria do not acquire genetic elements encoding for virulence factors, they remain benign commensals.

There are several subtypes of E. coli, however, that can cause infections and illnesses of varying severity. For example, classes of E. coli known as uropathogenic E. coli (UPEC) can cause urinary tract infections, the most common extra-intestinal E. coli infection. E. coli cause about 80 percent of all uncomplicated urinary tract infections, which account for more than 7,000,000 physician office visits per year in the United States. Similarly, menigitis-associated E. coli (MNEC) is the most common pathogen responsible for meningitis, particularly during the neonatal period. Neonatal meningitis can affect one out of every two to four thousand infants.

E. coli strains can also cause other illnesses. For example, if E. coli is translocated into the peritoneal cavity through a perforation or tear in the intestine, it may cause peritonitis, a potentially fatal infection. E. coli can also cause sepsis, an infection which results from bacteria entering the bloodstream. The bacteria are usually introduced into the bloodstream from some distal infection site, such as the kidney, skin, or lung. There are approximately 500,000 cases of septicemia per year in the U.S. and the mortality rate remains between 20 and 50 percent despite a substantial research effort into treatment for this condition. Approximately 45 percent of septicemia cases are due to gram-negative bacteria such as E. coli.

Certain virulent strains of E. coli can also cause various diarrheal diseases. The severity of diarrheal illness varies considerably and can be fatal. Even when not fatal, diarrheal disease is one of the foremost public health problems for infants and children in developing countries, despite recent advances in understanding of underlying causes and disease management.

Diarrhea is a particularly dangerous disease for children and infants. It is the leading cause of death in children under 5 years of age, accounting for 3 to 4 million deaths each year worldwide. Multiple episodes of acute diarrhea or persistent diarrhea can also seriously affect growth, nutritional status and cognition. Steiner, T. S., et al. Enteroaggregative Escherichia Coli Produce Intestinal Inflammation and Growth Impairment and Cause Interleukin-8 Release from Intestinal Epithelial Cells, J. Infect. Dis. 177:88-96 (1998).

Diarrheagenic E. coli strains have been divided into at least six major categories based on their epidemiological and clinical features, specific virulence determinants, and association with certain serotypes: enterotoxigenic E. coli (ETEC), enteroinvasive E. coli (EIEC), diffusely adherent E. coli (DAEC), enteropathogenic E. coli (EPEC), enterohemorrhagic E. coli (EHEC), and enteroaggregative E. coli (EAEC). Nataro, J., et al., Diarrheagenic Escherichia coli, Clin. Microbio. Rev. 11:142-201 (January 1998); Trung Vu Nguyen, etal., Detection and Characterization of Diarrheagenic Escherichia coli from Young Children in Hanoi, Vietnam, J. Clin. Microbio. 43:755-760, 755 (February 2005).

ETEC are specific strains of E. coli producing enterotoxins. Some enterotoxins are cytotoxic, damaging mucosal cells of the gastrointestinal tract, whereas others are merely cytotonic, inducing the secretion of water and electrolytes. ETEC is recognized as the primary cause of traveler's diarrhea. In contrast, EIEC are strains that produce invasion factors and cause colonic tissue distruction and inflammation. EIEC closely resemble Shigella and cause an invasive, dysenteric form of diarrhea in humans. DAEC strains are identified according to their ability to diffusely adhere to the surface of a cell. EPEC is the oldest recognized category of diarrheagenic E. coli and often causes a profuse watery diarrheal disease. EPEC is a leading cause of infantile diarrhea in developing countries. Unlike ETEC or EIEC, EPEC produce no recognizable toxins or invasion factors. Another group of E. coli strains are EHEC, also known as Shiga toxin-producing E. coli (STEC). EHEC excrete potent verotoxins or Shiga toxins, often causing hemorrhagic colitis or bloody diarrhea. Lastly, EAEC, which are known for their ability to attach to tissue culture cells in an aggregative manner, have been associated with persistent diarrhea, especially in developing countries.

The genus Shigella, also a member of the family Enterobacteriaceae, is closely related to Escherichia, but is anaerogenic and unable to ferment lactose. Thus, it is usually discernable from E. coli. There are four species in the genus: S. dysenteriae, S. flexneri, S. boydii, and S. sonnei. Many strains, including M90T, E22383 and E23507 exist within the species S. flexneri.

Infection by Shigella flexneri, commonly known as shigellosis or bacillary dysentery, is a leading cause of infant mortality in developing countries. There are approximately 165 million episodes of shigellosis each year, resulting in about 1.5 million deaths. Shigellosis is characterized by diarrhea, fever, vomiting and stomach cramps. In rare cases, young children with the disease can experience seizures. Shigellosis is particularly common and can cause recurrent problems in settings where hygiene is poor. Transmission of the disease occurs mainly by consumption of water contaminated by feces from infected individuals. The infection is very efficient, as ingestion of less than 10 bacterial cells is enough to cause an infection. Children, especially toddlers aged 2 to 4, are the most likely to contract shigellosis.

EPEC, EHEC and Shigella each use a similar mechism to invade and infect eurkaryotic cells and cause diarrhea. This mechanism is known as the type III secretory system (TTSS). Pathogens using this mechanism induce a characteristic histopathological lesion, termed attaching/effacing (A/E) lesion, defined by the intimate attachment of bacteria to the epithelial surface and the subsequent effacement of host cell microvilli. Sekiya, K., et al., Supermolecular Structure of the Enteropathogenic Escherichia coli Type III Secretion System and its Direct Interaction with the EspA-Sheath-like Structure, PNAS, 98:11,638-11,643 (2001). The TTSS enables the pathogens to inject virulence proteins directly into the cytoplasm of the eukaryotic host cells they infect. Yip, Calvin, et al., Structural Characterization of a Type-III Secretion System Filament Protein in Complex with its Chaperone, Nat. Structural & Mol. Biol. 12:75-81 (2005).

The TTSS apparatus consists of two distinct parts: (1) an elongated, hollow extracellular structure, often termed the needle, and (2) a cylindrical base, similar to the flagellar basal body which crosses the two bacterial membranes and ensures the stabilization of the whole structure upon the cell envelope. It is believed that the needle is physically linked to the basal body. Tampakaki, A., et al., Conserved Features of Type III Secretion, Cell. Microbio. 6:805-816 (2004).

The mechanism of TTSS is triggered when the pathogen comes into close contact with a host cell. While the mechanism described below corresponds specifically to EPEC, it is believed that a similar mechanism exists for all pathogens that express a TTSS. Sekiya, PNAS 98:11,638; Ebel, F., et al., Temperature- and Medium-Dependent Secretion of Proteins by Shiga Toxin-Producing Escherichia coli, Infect. lmm. 64:4472-4479 (November 1996).

The TTSS is believed to be a multistep mechanism resulting in eventual infection. First, a pair of ring-like structures situated on the inner and outer membranes of the bacteria are assembled. The bacteria then produces an injection port composed of E. coli secretion component F (EscF), which is anchored to the bacterial inner and outer membrane. Multipers of E. coli secreted protein A (EspA) then attach to the tip of the EscF. EspA subsequently initiates polymerization from the tip of the EscF and assembles a sheath-like structure, known as the needle. The needle is expandable, and its elongation is controlled by the amount of EspA secreted. The needle builds a physical bridge between the bacterium and the host cell membrane.

At the end of the needle, two E. coli secreted proteins B (EspB) and D (EspD), hetero-oligomerize into complexes which form pores in the eukaryotic target cell. These pores allows proteins to be injected into the cytoplasmic compartment of the epithelial cells. EspB and EspD, as well as the translocated intimin receptor (Tir), are then translocated into the host cells. It is known that the proper function of EspB is critical for the successful use of the type three secretory system by the pathogenic bacteria. By introducing the bacterial proteins into the host cell, the bacterium forces the cell to cooperate in its own infection. Additionally, as Tir is translocated, it is projected beyond the intestinal cell surface and directly binds with the bacteria adhesion molecule intimin. Intimin-Tir binding triggers polymerization of actin and other cytoskeletal components at the site of attachment, which then disrupts the normal enterocyte microvilli, forming the distinctive pedestal. This change is often referred to as the A/E lesion. After many bacterium have adhered to the intestinal cell lining in this manner, symptoms of the infection, such as diahrrea, commence. See FIG. 16 for a diagram of a typical TTSS.

In contrast to EPEC, EHEC and Shigella, EAEC are a classification of E. coli that do not express type-III secretory systems. Instead, the distinguishing feature of EAEC strains is their ability to attach to tissue culture cells in an aggregative manner. The bacteria align themselves in parallel rows to tissue cells. This aggregation has been described as “stacked brick-like”.

EAEC pathogenesis has three stages: (1) adherence to the intestinal mucosa by aggregative adherence fimbriae (AAF) or other adherent factors; (2) increased production of mucus by the bacteria and host cell, which is deposited as a mucus biofilm on the surface of the enterocyte; and (3) an inflammatory response with cytokine release, mucosal toxicity and intestinal secretion. Huang, D.B., et al., Enteroaggregative Escherichia coli: An Emerging Enteric Pathogen, Am. J. Gastroenterol. 99(2):383-389 (2004). EAEC include many strains of E. coli, including E. coli O42, O4, O:H10 and O44:H18.

EAEC strains, associated with persistent diarrhea in young children, are responsible for about 10% of the cases of diarrhea in children. In developing countries, EAEC strains are associated with between 8% and 32% of all acute pediatric diarrhea cases and with 20% to 30% of all persistent diarrhea. The strains have also been associated with traveler's diarrhea and acquired immunodeficiency syndrome (AIDS)-associated diarrhea. The duration of EAEC-associated diarrhea cases in children less than 3 years of age can average 17 days, which is longer than that associated with any other pathogen.

As a means for protecting children younger than 5 years of age against various diarrheal diseases, breasffeeding has been identified as the most effective intervention. Multiple studies have shown that exclusive breasffeeding and, to a lesser extent, partial breasffeeding, can protect against acute and persistent diarrhea. Victoria, C. G., et al., Risk Factors for Deaths Due to Respiratory Infections Among Brazilian Infants, Int. J. Epidemiol. 18: 918-25 (1989). The effectiveness of breasffeeding as a protective intervention can be attributed to the multiple anti-infective, anti-inflammatory and immunoregulatory factors transmitted through human milk. Newburg, D. S. Human Milk Glycoconjugaes that Inhibit Pathogens, Curr. Med. Chem. 6: 117-127 (1999).

Lactoferrin, an iron-binding glycoprotein, is one of the major multifunctional agents present in human milk. It is also found in exocrine secretions, such as tears, saliva, and those from specific cell types such as neutrophils. Human lactoferrin has been reported to protect against Gram-negative bacteria in a variety of ways.

Though not wishing to be bound to this or any theory, it is believed that human lactoferrin exerts a bacteriostatic activity by depriving the microorganism of the iron that is necessary for its growth. Thus, by sequestering the environmental iron that is essential for the growth of pathogenic microorganisms, human lactoferrin effectively inhibits the growth of those microorganisms.

It is believed that human lactoferrin can also exert a bacteriocidal activity due to its direct binding to the microbial membrane. Human lactoferrin binds to the lipid A portion of lipopolysaccharide (LPS) on the bacterial cell surface which disrupts the bacterial cell membrane and alters its permeability. It is speculated that the binding of human lactoferrin to LPS can interfere with type III secretory machinery, causing the secreted proteins to be released and then digested. Ochoa, T. J., et al., Lactoferrin Impairs Type III Secretory System Function in Enteropathogenic Escherichia coli, Infect. Immun. 71:5149-5155 (2003).

Several studies have examined the effect of human lactoferrin on various bacterial species. For example, a 2003 study investigated the effect of recombinant human lactoferrin on TTSS. Recombinant human lactoferrin is known to be very similar in amino acid sequence to native human lactoferrin, but has been described to have a different glycosylation pattern. The study demonstrated that recombinant human lactoferrin can impair the function of the type III secretory system in enteropathogenic E. coli. Id. The study also found, however, that human lactoferrin did not impair the growth of EPEC. Id.

A 2001 study demonstrated that human lactoferrin can inhibit the adhesion of EPEC to HeLa cells. Nascimento de Araujo, A., et al., Lactoferrin and Free Secretory Component of Human Milk Inhibit the Adhesion of Enteropathogenic Escherichia coli to HeLa Cells, BMC Microbiol. 1:25 (2001). This study did not address the ability of human lactoferrin to inhibit the growth of microorganisms.

While human lactoferrin appears to have a positive effect on the symptoms of diarrheal diseases, some women are unwilling or unable to breasffeed. As such, it would be useful to provide an alternative to human milk that would be effective in preventing or eliminating diarrheal infections. Such an alternative should also be effective in inhibiting the growth bacterial pathogens that cause diarrheal infections.

### SUMMARY OF THE INVENTION

Briefly, therefore, the present invention is directed to a novel method for inhibiting the growth of Enterobacteriaceae in a subject and/or preventing or treating an infection caused by Enterobacteriaceae, the method comprising administering to the subject an effective amount of bovine lactoferrin.

The present invention is also directed to a novel method for inhibiting the growth of bacterial pathogens expressing a type III secretory system in a subject and/or preventing or treating an infection caused by bacterial pathogens expressing a type III secretory system. The method comprises administering to the subject an effective amount of bovine lactoferrin. In certain embodiments, these pathogens can be selected from the group consisting of Salmonella, Shigella, Yersinia, Pseudomonas, and Escherichia.

In addition, the present invention is directed to a novel method for inhibiting the adherence of bacterial pathogens expressing a type III secretory system to human intestinal cells, the method comprising contacting the pathogens with an effective amount of bovine lactoferrin.

The present invention is additionally directed to a novel method for causing the premature release of EspB in a bacterial pathogens expressing a type III secretory system as well as a method for causing the degradation of EspB in a bacterial pathogens expressing a type III secretory system. The method comprises contacting the pathogens with an effective amount of bovine lactoferrin.

Further, the present invention is directed to a novel method for inhibiting the growth of enteroaggregative E. coli in a subject as well as preventing or treating an infection caused by enteroaggregative E. coli, the method comprising administering to the subject an effective amount of bovine lactoferrin.

The present invention is also directed to a novel method for inhibiting the adherence of enteroaggregative E. coli to intestinal cells, the method comprising contacting the enteroaggregative E. coli with bovine lactoferrin.

In addition, the present invention is directed to a novel enteral formulation or infant formula comprising bovine lactoferrin which has been isolated from whole milk and has a low somatic cell count. In certain embodiments, the enteral formulation further contains casein glycomacropeptide.

Among the advantages of the present invention is that it provides a non-pharmaceutical remedy for many bacterially-mediated diseases and infections. The bLF can be easily supplemented into an enteral composition or infant formula to prevent the onset of or treat such illnesses. Alternatively, the bLF of the present invention can be administered onto the surface of food products or intermixed with the food products to inhibit the growth of bacterial pathogens.

### BRIEF DESCRIPTION OF THE DRAWINGS

For a more complete understanding of the present invention, reference is now made to the following descriptions taken in conjunction with the accompanying drawings.

FIG. 1 illustrates the bacteriostatic effect of bLF on the growth of EPEC.

FIG. 2 illustrates the bacteriostatic effect of bLF on the growth of EAEC.

FIG. 3 illustrates the bacteriostatic effect of bLF on the growth of Shigella.

FIG. 4 illustrates the effect of varying concentrations of bLF on the inhibition of growth of EPEC.

FIG. 5 illustrates the effect of varying concentrations of bLF on the inhibition of growth of EAEC.

FIG. 6 illustrates the effect of varying concentrations of bLF on the inhibition of growth of Shigella.

FIG. 7 illustrates the effect of saturating concentrations of iron on the ability of bLF to inhibit the of growth of EPEC.

FIG. 8 illustrates the effect of saturating concentrations of iron on the ability of bLF to inhibit the of growth of EAEC.

FIG. 9 illustrates the effect of saturating concentrations of iron on the ability of bLF to inhibit the of growth of Shigella.

FIG. 10 illustrates the effect of various bLF preparations on the growth inhibition of EPEC.

FIG. 11 illustrates the effect of various bLF preparations on the growth inhibition of EAEC.

FIG. 12 illustrates the effect of various bLF preparations on the growth inhibition of Shigella.

FIG. 13 illustrates the adherence of EAEC to HEp-2 cells in the absence of bLF.

FIG. 14 illustrates the effect of bLF on the adherence of EAEC to HEp-2 cells.

FIG. 15 illustrates the effect of saturating concentrations of iron on the ability of bLF to inhibit the adherence of EAEC to HEp-2 cells.

FIG. 16 is a diagram of the type III secretory system.

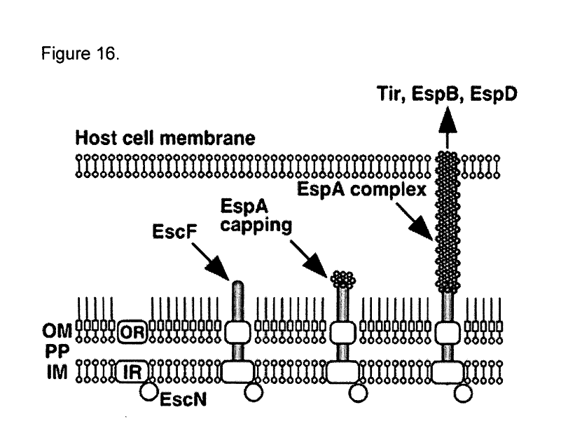

FIG. 17 is a western blot of STEC HW1 and STEC 306-7, alone and in the presence of bovine and human lactoferrin.

FIG. 18 is a western blot of STEC TWO 8023 and C600, alone and in the presence of bovine and human lactoferrin.

### DETAILED DESCRIPTION OF THE PREFERRED EMBODIMENTS

Reference now will be made in detail to the embodiments of the invention, one or more examples of which are set forth below. Each example is provided by way of explanation of the invention, not a limitation of the invention. In fact, it will be apparent to those skilled in the art that various modifications and variations can be made in the present invention without departing from the scope or spirit of the invention. For instance, features illustrated or described as part of one embodiment, can be used on another embodiment to yield a still further embodiment.

Thus, it is intended that the present invention covers such modifications and variations as come within the scope of the appended claims and their equivalents. Other objects, features and aspects of the present invention are disclosed in or are obvious from the following detailed description. It is to be understood by one of ordinary skill in the art that the present discussion is a description of exemplary embodiments only, and is not intended as limiting the broader aspects of the present invention.

The term “probiotic” means a microorganism that exerts beneficial effects on the health of the host.

The term “prebiotic”, as used herein, means a non-digestible food ingredient that stimulates the growth and/or activity of probiotics.

The term “subject” means any mammal, preferably a human. In certain embodiments, the subject is in need of prevention or treatment of an infection caused by Enterobacteriaceae or bacterial pathogens expressing a TTSS. In other embodiments, the subject is in need of inhibition of the growth of bacterial pathogens expressing a TTSS. In particular embodiments, the subject is in need of inhibition of the growth of EAEC or is in need of prevention or treatment of an infection caused by EAEC. In certain embodiments, the subject is a child or infant. In some embodiments, the subject is an infant who does not receive human breast milk.

The term “inhibiting” means to decrease, limit, or block the growth or action of one or more organisms.

As used herein, the term “treating” means ameliorating, improving or remedying a disease, disorder, or symptom of a disease or condition.

The term “preventing” means to stop or hinder a disease, disorder, or symptom of a disease or condition through some action.

The terms “effective amount” refer to an amount that results in an inhibition of the growth of a pathogenic organism or an improvement or remediation of a disease, disorder, or symptoms of a disease or condition caused by the organism.

The term “infection” means invasion by and multiplication of pathogenic microorganisms in a subject's body, which may produce subsequent tissue injury and progress to overt disease through a variety of cellular or toxic mechanisms.

The terms “somatic cell count” mean a measure of the white blood cell count in milk.

The terms “enteral formulation” mean a composition for enteral administration to a subject, preferably through ingestion.

As used herein, the term “infant formula” means a composition that satisfies the nutrient requirements of an infant by being a substitute for human milk. In the United States, the contents of an infant formula are dictated by the federal regulations set forth at 21 C.F.R. Sections 100, 106, and 107. These regulations define macronutrient, vitamin, mineral, and other ingredient levels in an effort to stimulate the nutritional and other properties of human breast milk.

In accordance with the present invention, a novel method for inhibiting the growth of Enterobacteriaceae in a subject has been discovered. The method comprises administering to the subject an effective amount of bovine lactoferrin (bLF). Bovine lactoferrin is a glycoprotein that belongs to the iron transporter or transferrin family. It is isolated from bovine milk, where it is found as a component of whey.

There are known to be significant differences between the amino acid sequence, glycosylation patterns and iron-binding capacity in human and bovine lactoferrin. Additionally, there are multiple and sequential processing steps involved in the isolation of bovine lactoferrin from cow's milk which are expected to result in physiochemical properties of the resulting bovine lactoferrin preparation. Human and bovine lactoferrin are also reported to have differences in their abilities to bind the lactoferrin receptor found in the human intestine. Specifially, bovine lactoferrin is known to have a poor ability to bind the lactoferrin receptor of the human intestine. Because human and bovine lactoferrin are so different in these respects, the results obtained in the present invention, using bovine lactoferrin, were unexpected and surprising.

As discussed above, it was believed that the main mechanism behind the bacteriostatic effect of human and presumably bovine lactoferrin was iron sequestration from the pathogens. In contrast, the present invention has shown that bovine lactoferrin's bacteriostatic effect is iron saturation independent. Thus, the bacteriostatic effect of bovine lactoferrin is believed to occur through a different mechanism than that of human lactoferrin.

There are several studies dealing with the bacteriostatic effects of bLF. For example, one study found that bLF has a bacteriostatic effect on the proliferation of various species of Clostridium in mice. Teraguchi, S., et al., Bacteriostatic Effect of Orally Administered Bovine Lactoferrin on Proliferation of Clostridium Species in the Gut of Mice Fed Bovine Milk, Appl. Environ. Microbiol. 61:501-506 (1995). bLF has also been demonstrated to have a bacteriostatic effect on intestinal Enterobacteriaceae in mice. Teraguchi, S., et al., The Bacteriostatic Effect of Orally Administered Bovine Lactoferrin on Intestinal Enterobacteriaceae of SPF Mice Fed Bovine Milk, Biosci. Biotech. Biochem. 58:482-487 (1994). Though the predominant species of Enterobacteriaceae that was isolated in the study was E. coli, the study provides no evidence of the growth-inhibiting effect of bLF on bacterial pathogens having type III secretory systems or specific E. coli classifications such as EAEC.

In contrast to the above studies, however, a similarly-conducted study found that bLF had no growth-inhibiting effect on enterotoxigenic E. coli. Sarelli, L., et al., Lactoferrin to Prevent Experimental Escherichia coli Diarrhea in Weaned Pigs, Intl. J. Appl. Res., available at http://www.jarvm.com/articles/Vol1lss4/Heinonen.htm. The study also demonstrated no significant reduction in the occurrence of experimentally-induced diarrhea or hemolytic E. coli counts in feces. Id.

Several patents also relate to the effects of bLF on various bacteria and/or illnesses. For example, U.S. Patent App. No. 20030203839 to Kruzel, et al. relates to the use of bLF to treat the. progression of systemic inflammatory response syndrome (SIRS) into sepsis, severe sepsis, septic shock and multiple organ failure.

U.S. Patent Apps. No. 20040043922 to Naidu and 20030229011 to Braun relate to a method for reducing microbial contamination on food products. The method involves treating a food product with immobilized lactoferrin. While the invention is useful in treating EPEC and Shigella spp., it requires that the lactoferrin be immobilized to be effective.

U.S. Patent App. No. 20040152624 to Varadhachary, et al. relates to a method for treating bacteremia by administering an effective amount of bLF to a subject. According to the reference, the method also causes decreasing levels of circulating bacteria. None of these references, however, disclose the benefits of bLF on bacterial pathogens having type III secretory systems. Similarly, none of the references discloses the benefits of bLF on EAEC. Thus, the present invention provides surprising benefits over the references discussed above.

In particular embodiments of the present invention, the Enterobacteriaceae can comprise E. coli and Shigella. In another embodiment of the invention, the Enterobacteriaceae can comprise enteropathogenic E. coli, enteroaggregative E. coli and Shigella. In yet another embodiment of the invention, the Enterobacteriaceae can comprise E. coli E2348/69, E. coli O42 and Shigella flexneri M90T. The strains E. coli E2348/69, E. coli O42 and Shigella flexneri M90T are important pediologically, as they cause many deleterious infections in infants and children.

In other embodiments of the invention, the growth of bacterial pathogens expressing a type III secretory system is inhibited by an effective amount of bLF. In certain embodiments, the bacterial pathogens expressing a type III secretory system can comprise Salmonella, Shigella, Yersinia, Pseudomonas and Escherichia. In an embodiment, the Escherichia pathogen can comprise enteropathogenic E. coli or enterohemorrhagic E. coli. EPEC include many strains of E. coli, including E. coli E2348/69, O26, O55, O157, O111, O114:H2, O119:H6, O126, O127:H6, O128:H2 and O142:H6. Similarly, the EHEC can comprise strains STEC TWO 8023, STEC HW1, STEC 306-7, O157:H7, O111:H8, O104:H21, O111, O26 or O26:H111.

In a separate embodiment of the method of the present invention, the method prevents or treats an infection caused by Enterobacteriaceae in a subject. This includes any infections known in the art to be caused by Enterobacteriaceae. More specifically, this includes any infections known in the art to be caused by enteropathogenic E. coli, enteroaggregative E. coli or Shigella flexneri. It also includes all infections known in the art to be caused by a bacterial pathogen expressing a type III secretory system. In an embodiment of the present invention, the infection can include a urinary tract infection, neonatal meningitis, peritonitis, shigellosis, or any gastrointestinal infection or disease.

In another embodiment of the invention, the growth of pathogenic bacteria is inhibited without a corresponding increase in levels of toxins produced by the bacteria. It is known that growth inhibition is a trigger for the induction of bacteriophages that carry Stx genes. Many antibiotics are known to increase toxin production via induction of a phage lytic cycle. The present invention was surprising in that it did not induce an increased level of toxin production during simultaneous growth inhibition. Thus, the present invention can inhibit the growth of pathogenic bacteria without simultaneously increasing toxin production. This unexpected benefit is discussed further in Example 4.

In certain embodiments of the invention, the bLF has been isolated from whole milk. In certain other embodiments, the bLF has a low somatic cell count. In some embodiments, the bLF has both been isolated from whole milk and has a low somatic cell count.

Though not wishing to be bound by this or any theory, it is believed that bLF which has been isolated from whole milk has less lipopolysaccharide (LPS) initially bound than does bLF that has been isolated from milk powder. Additionally, it is believed that bLF with a low somatic cell count has less initially-bound LPS. Because this form of bLF has less initially-bound LPS, more binding sites are available on its surface. This is thought to aid bLF in binding to the appropriate location and disrupting the infection process.

The bLF that is used in certain embodiments of the present invention can be any bLF isolated from whole milk and/or having a low somatic cell count. By way of example, suitable bLF is available from Tatua Co-operative Dairy Co., Ltd., in Morrinsville, New Zealand.

In an embodiment of the present invention, the growth-inhibiting effect of the bLF is dose-dependent. As the dose of bLF is increased, the growth-inhibiting effect of the bLF is also increased.

In another embodiment of the present invention, the growth-inhibiting effect of the bLF is dependent on the concentration of iron present in the intestine of the subject. The bLF of the present invention loses its bacteriostatic capabilities after being saturated with iron.

In the present invention, bLF has a growth-inhibitory effect on bacterial pathogens that have type III secretory systems as well as EAEC, a pathogen that lacks a type III secretory system. The present invention illustrates that while bLF is effective on bacterial pathogens expressing a type III secretory system, it is not necessarily specific to the type III secretory system. bLF is also effective in inhibiting growth of non-TTSS pathogens. Though not wishing to be bound to this or any theory, it is believed that the membrane-anchored virulence proteins of EAEC, which are important for adherence, may be disrupted by bLF interacting with the bacterial surface. There are several adherence proteins that could be responsible for bLF's effect on EAEC adherence, including AAF/I and AAF/II. Thus, bLF can protect infants from gastrointestinal infection by preventing the attachment of EAEC to intestinal epithelia and thereby protecting it from colonization by pathogens.

In a particular embodiment of the method of the invention, bLF can inhibit the adherence of EAEC to intestinal cells. In this embodiment, the anti-adherence effect is not affected by the concentration of iron present in the intestine of the subject.

An effective amount of bLF for use in the present invention can be between about 0.001 mg and about 100 g per day. In an embodiment, an effective amount of bLF can be between about 0.1 mg and 10 g per day. In another embodiment, an effective amount of bLF can be between about 10 mg and 1500 mg per day. In a particular embodiment of the invention, the bLF can be administered in three daily doses.

In various embodiments, the bLF may be administered via a solution, capsule, tablet, or caplet. Carriers for bLF can have a bLF concentration of between about 0.01% to about 100%.

The present invention also encompasses a method for inhibiting the growth of EPEC, EAEC or Shigella on a food product, the method comprising treating the food product with an effective amount of bLF. In this embodiment, the bLF can be sprayed onto the surface of the food or can be intermixed with the ingredients of the food product.

In another embodiment of the present invention, the invention is an enteral formulation comprising casein glycomacropeptide and bovine lactoferrin which has been isolated from whole milk and has a low somatic cell count.

In an embodiment, the enteral formulation also includes a long-chain polyunsaturated fatty acid (LCPUFA). Suitable LCPUFAs may include, but are not limited to, a-linoleic acid, y-linoleic acid, linoleic acid, linolenic acid, eicosapentanoic acid (EPA), arachidonic acid (ARA) and docosahexaenoic acid (DHA). In one method of the present invention, an effective amount of LCPUFA may correspond to between about 3 mg per kg of body weight per day to about 150 mg per kg of body weight per day. In one embodiment of the invention, the amount is from about 6 mg per kg of body weight per day to about 100 mg per kg of body weight per day. In another embodiment the amount is from about 10 mg per kg of body weight per day to about 60 mg per kg of body weight per day.

The enteral formulation may also contain a probiotic. Any probiotic known in the art will be acceptable in this embodiment. In a particular embodiment, the probiotic is chosen from the group consisting of Lactobacillus and Bifidobacterium. In this embodiment, the probiotic can be Lactobaccilus rhamnosus GG (LGG) or Bifidobacterium lactis (Bb-12).

In yet another embodiment of the invention, the enteral formulation may contain a prebiotic. Any prebiotic known in the art will be acceptable in this embodiment. Prebiotics may include lactulose, galacto-oligosaccharide, fructo-oligosaccharide, isomalto-oligosaccharide, soybean oligosaccharides, lactosucrose, xylo-oligosacchairde, and gentio-oligosaccharides.

In another embodiment, the invention is an infant formula comprising bLF which has been isolated from whole milk and has a low somatic cell count. The infant formula may contain casein glycomacropeptide. The infant formula may also contain at least one LCPUFA. In yet another embodiment, the infant formula may contain at least one probiotic and/or a prebiotic. In one particular embodiment, the infant formula contains bLF which has been isolated from whole milk and has a low somatic cell count, casein glycomacropeptide, at least one LCPUFA, at least one probiotic, and at least one prebiotic.

The infant formula for use in the present invention is nutritionally complete and contains suitable types and amounts of lipid, carbohydrate, protein, vitamins and minerals. The amount of lipid or fat typically can vary from about 3 to about 7 g/100 kcal. The amount of protein typically can vary from about 1 to about 5 g/100 kcal. The amount of carbohydrate typically can vary from about 8 to about 12 g/100 kcal. Protein sources can be any used in the art, e.g., nonfat milk, whey protein, casein, soy protein, hydrolyzed protein, amino acids, and the like. Carbohydrate sources can be any used in the art, e.g., lactose, glucose, corn syrup solids, maltodextrins, sucrose, starch, rice syrup solids, and the like. Lipid sources can be any used in the art, e.g., vegetable oils such as palm oil, soybean oil, palmolein, coconut oil, medium chain triglyceride oil, high oleic sunflower oil, high oleic safflower oil, and the like.

Conveniently, commercially available infant formula can be used. For example, Enfamil®, Enfamil® Premature Formula, Enfamil® with Iron, Lactofree®), Nutramigen®, Pregestimil®, and ProSobee® (available from Mead Johnson & Company, Evansville, Ind., U.S.A.) may be supplemented with suitable levels of bLF, casein glycomacropeptide, LCPUFA(s), probiotic(s), and/or prebiotic(s) and used in practice of the method of the invention.

The following examples describe various embodiments of the present invention. Other embodiments within the scope of the claims herein will be apparent to one skilled in the art from consideration of the specification or practice of the invention as disclosed herein. It is intended that the specification, together with the examples, be considered to be exemplary only, with the scope and spirit of the invention being indicated by the claims which follow the examples. In the examples, all percentages are given on a weight basis unless otherwise indicated.

### EXAMPLE 1

This example describes the materials and methods necessary to show the effect of bovine lactoferrin on bacterial pathogens having type III secretory systems. Three strains of bacteria were utilized in the present invention. The strains included STEC HW1, STEC 306-7 and STEC TWO 8023. Each of these strains is a different organism that utilizes a TTSS to infect cells. The bacterial strain C600, a pathogen that does not utilize a TTSS, was used as a negative control in the testing.

Bacteria from overnight growth in Luria Broth were inoculated at 1:50 dilution in Dulbecco's modified Eagle's medium (DMEM) with 25 mM HEPES, pH 7.4, and incubated at 37° C. in a 5% CO₂ incubator without shaking. A 5 mL aliquot was measured into labeled tubes and bacterial growth was monitored spectrophotometrically at OD₆₀₀ every hour for seven hours. Colony forming units (cfu) were evaluated during the logarithmic growth phase (4 hours) in all the control samples without bLF.

In order to eliminate the possibility that the anti-TTSS activity observed for bovine lactoferrin was due to iron sequestration, the bovine lactoferrin used in this experiment was saturated with iron through incubation of the lactoferrin with 5 times the iron necessary to saturate all the iron binding sites in the lactoferrin preparation.

At each hour during the experiments, the sample was centrifuged at 13200 rpm for 10-15 minutes to separate cell associated from released components. The supernatants, representing the non-cell associated material (that released from the bacteria), were retained and placed in 0.1 ml of SDS sample buffer and heated at 100° C. for 5 minutes. The pellets from this centrifugation step were washed in phosphate buffered saline and resuspended in 0.1 ml SDS sample buffer and heated at 100° C. for 5 minutes. The supernatants and pellets, both in sample buffer, were then resolved by SDS-polyacrylamide gel electrophoresis using customary methods. The resulting gel was then transferred to a membrane, such as polyvinyidiflouride or nitrocellulose, using common methods. The presence of EspB in the supernatants (released proteins) and pellets (cell associated proteins) was then detected by Western blotting using antibodies specific to EspB.

In this example, the bLF was obtained from Tatua Co-operative Dairy Co., Ltd, Morrinsville, New Zealand. Unless otherwise noted, all experiments were conducted using a concentration of 10 mg/mL (0.125 mM) or 1 mg/mL of bLF, which are approximately the concentrations present in human colostrum and mature milk, respectively.

Data are expressed as mean i standard deviation (SD) unless otherwise noted. For analysis of the growth inhibitory effect of lactoferrin, two regression lines were calculated and the difference between the two regression slopes were then tested using t statistics (Armitage P, 1980). Paired data were then analyzed by Wilcoxon Rank-Sum test for differences in medians. For analysis of the iron content of various lactoferrin preparations and their growth inhibitory effect, a linear regression was calculated and expressed as R-squared for each bacteria.

### EXAMPLE 2

This example illustrates the effect of bovine lactoferrin on the attachment of bacterial pathogens to eukaryotic cells. Specifically, this example illustrates the effect of bovine lactoferrin on the protein EspB in the type III secretory system.

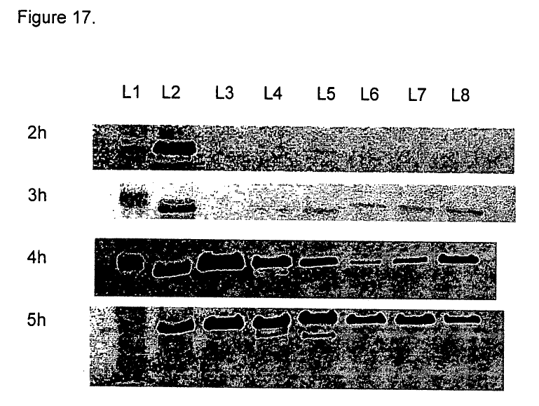

The western blot shown in FIG. 17 illustrates the effect of bovine and human lactoferrin on STEC HW1 and STEC 306-7. Lane L1 represents the molecular weight markers. Lane L2 represents purified EspB to indicate the appropriate position of this protein in the Western blot. Lane L3 in each of the gel pictures represents the amount of EspB in the supernatant (released) after 2, 3, 4 or 5 hours of culture growth of strain HW1. Lane L4 represents EspB in the supernatant of a 2, 3, 4 or 5 hour culture of strain HW1 which has been growing in 1 mg/mL human recombinant lactoferrin. Lane L5 represents EspB in a supernatant of a 2, 3, 4 or 5 hour culture of strain HW1 which has been growing in 1 mg/mL bovine lactoferrin. Lane L6 represents EspB supernatant of a 2, 3, 4 or 5 hour culture of strain 306-7. Lane L6 represents EspB supernatant of a 2, 3, 4 or 5 hour culture of strain 306-7 which has been growing in 1 mg/mL human recombinant lactoferrin. Lane L6 represents EspB supernatant of a 2, 3, 4 or 5 hour culture of strain 306-7 which has been growing in 1 mg/mL bovine lactoferrin.

The western blot shown in FIG. 17 illustrates that in the absence of bLF (lanes L3 and L6), EspB is not observed in the supernatant (not released from the bacteria) until after 4 hours of growth. This illustrates the normal course of production and release of the TTSS components during undisturbed growth of the bacteria. In contrast, in the presence of bLF EspB is detected in the supernatant earlier (3 hours, lanes L5 and L8), than in the absence of bLf, indicating premature release of EspB. This finding is important because EspB is an essential component required for successful use of the TTSS, and premature release would limit the effectiveness of the TTSS for infection. The same effect is not seen with a preparation of human lactoferrin (lanes L4 and L7). In addition to premature release of EspB, this experiment also provides evidence that bLf is acting as a protease to degrade EspB (and therefore not allow it to be reused for formation of the secretory needle complex). This is indicated by the numerous bands identified as EspB (because it is recognized by the anti-EspB antibody) immediately under the position of the intact EspB protein in the gel (indicating generation of smaller EspB peptides). This phenomenon is observed for all strains expressing the TTSS, but not for the C600, a TTSS negative pathogen. It is known that different pathogens utilizing the TTSS express variants of EspB, providing an explanation for the variable degradation of this protein from each pathogen by bLf.

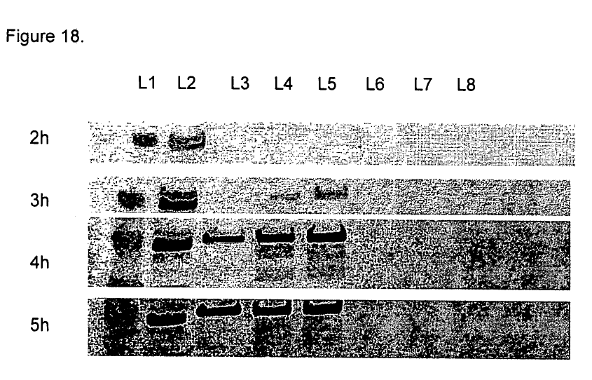

The western blot shown in FIG. 18 is organized in the same manner as that of FIG. 17 except that the strains tested are STEC TWO 8023 and C600, a negative control. The results of the western blot shown in FIG. 18 are also similar to those shown in FIG. 17. EspB is present in the media at 3 hours of growth in lactoferrin while it does not show up in the control until 4 hours. At both 4 and 5 hour time points, bovine lactoferrin caused the EspB to degrade. In addition, with three STEC strains evaluated at the 3 hour time point, bovine lactoferrin caused the EspB to be released into the medium more quickly than recombinant human lactoferin did. In addition, this figure provides additional evidence of degradation of EspB by bLf, as discussed for the previous figure.

Therefore, it is believed that the presence of bovine lactoferrin causes both a premature release and a degradation of EspB. EspB is required for changes in short circuit current across polarized intestinal epithelial cells and for membrane depolarization in Caco-2 cells. EspB is also required for induction of NF-KB activation, for interleukin-8 secretion, for transepithelial migration of neutrophils, and for a decrease in transepithelial electrical resistance, all of which may contribute to diarrheal disease. As such, a premature release and/or degradation of EspB may reduce or eliminate the symptoms of diarrheal disease.

### EXAMPLE 3

It is critical to the validity of this experiment that the bLf used in this experiment is iron saturated, and therefore not acting bacteriostatically to inhibit growth of the bacterial strains. This would alter the amount of EspB in each lane, potentially confounding the interpretation of the data.

This example illustrates the equivalent rate of growth of the bacteria in the presence or absence of bovine lactoferrin when saturated with iron. Because the growth of each culture in this experiment is similar either in the presence or absence of bLf, the observed effects on EspB are due to a novel activity of bLf and not to either growth inhibition or iron sequestration.

### EXAMPLE 4

This example illustrates the effect of bovine lactoferrin on the upregulation of Shiga-toxin (Stx) production during the growth inhibition of STEC. In addition to the type III secretory system, EHEC strains have additional virulence factors that contribute to their pathogenic personalities. One of the major virulence factors is the production of Stx, a toxin that inhibits protein synthesis. There are two forms of Stx: Stx1, which is almost identical to the toxin from Shigella dysenteriae type 1, and Stx2, which shares only 50 to 60% amino acid homology with Stx1. Both Stx1 and Stx2 have been associated with very serious disease. It is believed that Stx, which is released by bacteria residing in the intestinal lumen, is responsible for diarrheal disease, hemorrhagic colitis, and hemolytic-uremic syndrome. The toxin traverses the intestinal epithelial barrier, enters the bloodstream, and damages vascular cells of the colon, kidneys and the central nervous system.

It is known that growth inhibition is a trigger for the induction of bacteriophages that carry Stx genes. For example, multiple antibiotics including ciproflaxin, norfloxacin, trimethoprimsulfamethoxazole, ampicillin, fosfomycin, furazolidone, mitomycin, cefepime, and tetracycline are known to increase toxin production via induction of a phage lytic cycle. Animal models and clinical data in humans suggest that such toxin induction is central to the pathogenesis of the human disease.

Therefore, this study evaluated the effect of bovine lactoferrin on toxin production. Twenty strains of STEC were studied. The amount of toxin produced under growth inhibitory conditions of lactoferrin was determined and was compared to ciprofloxacin, a known inducer of toxin production. In the absence of lactoferrin or ciprofloxacin, the amount of Stx toxin produced by STEC was 35±14ng/well. In the presence of ciprofloxacin, the amount of Stx toxin produced was 682±193ng/well, an increase of nearly twenty-fold. In the presence of bovine lactoferrin, however, the amount of Stx toxin produced was only 35±14ng/well. Thus, bovine lactoferrin, unlike many other agents that impair bacterial growth, does so without induction of Stx toxin production. Bovine lactoferrin does not increase the production of toxins by bacterial pathogens when grown under the same conditions as the bacterial pathogens having no bovine lactoferrin. Likewise, in phage induction assays, no STEC strain could be induced to go into a lytic cycle when grown with a wide range of bovine lactoferrin concentrations.

### EXAMPLE 5

This example illustrates the effect of bovine lactoferrin on the growth of Enterobacteriaceae.

Three strains of bacteria were utilized in the present invention. The strains included EPEC E2348/69, EAEC O42, and Shigella flexneri M90T. Bacteria from overnight growth in Luria Broth were inoculated at 1:50 dilution in Dulbecco's modified Eagle's medium (DMEM) with 25 mM HEPES, pH 7.4, and incubated at 37° C. in a 5% CO₂ incubator without shaking. Bacterial growth was monitored spectrophotometrically at OD₆₀₀ every hour for seven hours. Colony forming units (cfu) were evaluated during the logarithmic growth phase (4 hours) in all the control samples without bLF.

In this example, the bLF was purchased from Tatua Co-operative Dairy Co., Ltd, Morrinsville, New Zealand. Unless otherwise noted, all experiments were conducted using a concentration of 10 mg/mL (0.125 mM) or 1 mg/mL of bLF, which are approximately the concentrations present in human colostrum and mature milk, respectively.

Data are expressed as mean ±standard deviation (SD) unless otherwise noted. For analysis of the growth inhibitory effect of lactoferrin, two regression lines were calculated and the difference between the two regression slopes were then tested using t statistics (Armitage P, 1980). Paired data were then analyzed by Wilcoxon Rank-Sum test for differences in medians. For analysis of the iron content of various lactoferrin preparations and their growth inhibitory effect, a linear regression was calculated and expressed as R-squared for each bacteria.

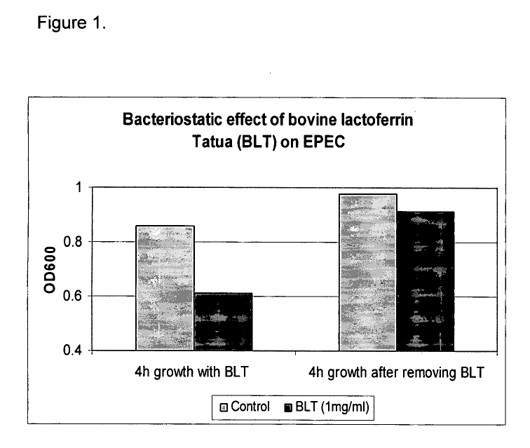

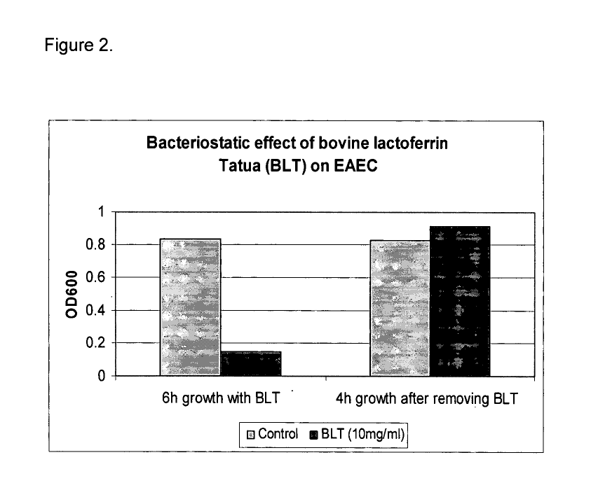

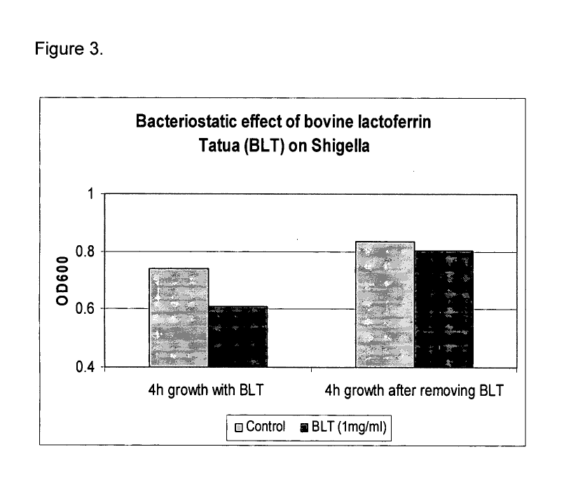

The bLF-treated bacteria (EPEC E2348/69, EAEC O42, and Shigella flexneri M90T) initially had a significant growth inhibition (OD₆₀₀ 0.149 vs. 0.831, lactoferrin vs. control, respectively). After removing the bLF from the media, the bacteria had normal growth (OD₆₀₀ 0.911 vs. 0.828, lactoferrin vs. control, respectively). This result indicates that bLF has an inhibitory effect on the growth of EPEC, EAEC and Shigella. This result also indicates that bLF has a bacteriostatic, and not bacteriocidal, effect on EPEC, EAEC and Shigella, as the bacteria returned to normal growth after removal of bLF from the media. These results are shown in FIGS. 1-3.

### EXAMPLE 6

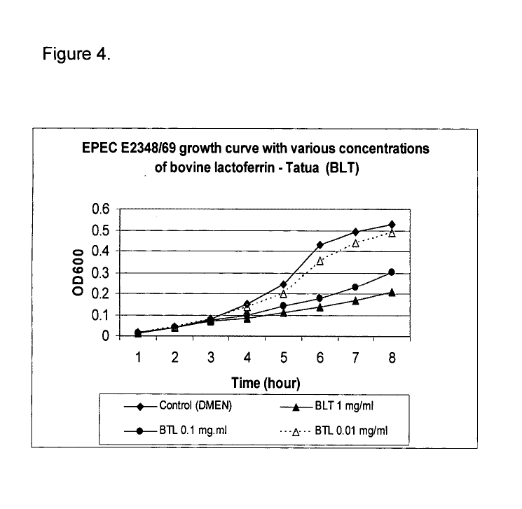

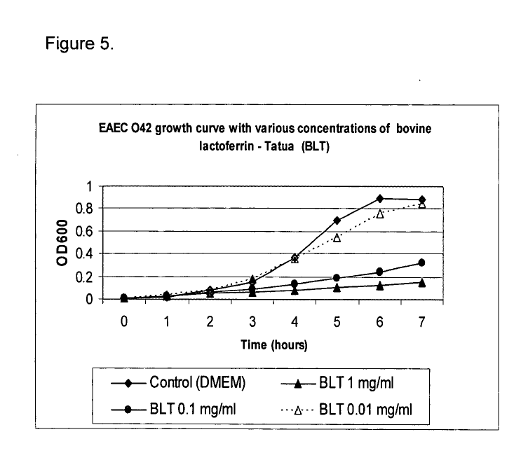

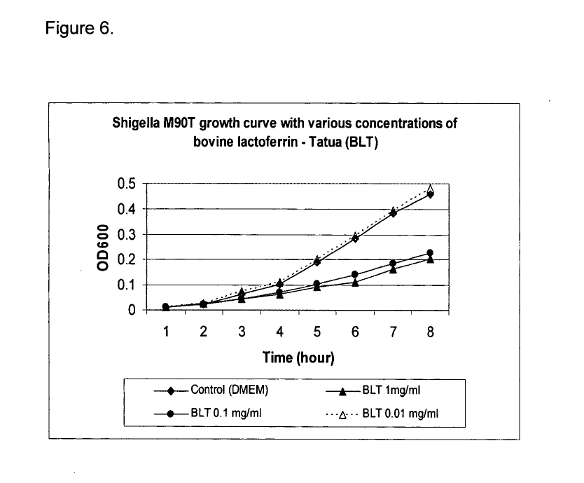

This example illustrates the effect of concentration and time on the growth inhibitory effect of bLF with regard to EPEC, EAEC and Shigella. In this example, the bLF was purchased from Tatua Co-operative Dairy Co., Ltd, Morrinsville, New Zealand. The bacteria were incubated in DMEM culture medium in the presence of bLF preparations at 1 mg/mL, 0.1 mg/mL and 0.01 mg/mL. FIGS. 4-6 show that bLF inhibited EPEC, EAEC and Shigella growth in a dose-dependent manner. As the concentration of bLF increased, its growth-inhibiting effect also increased.

### EXAMPLE 7

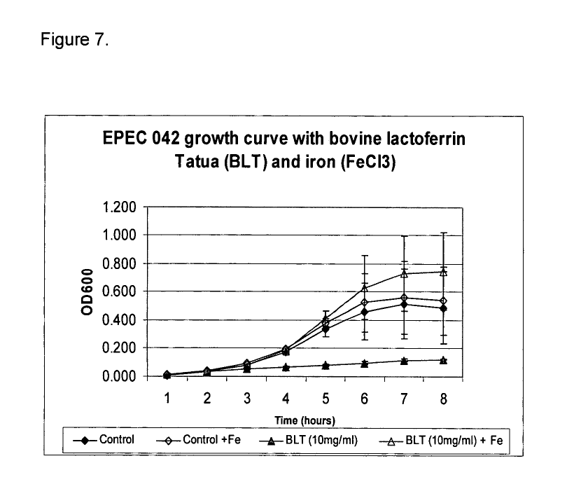

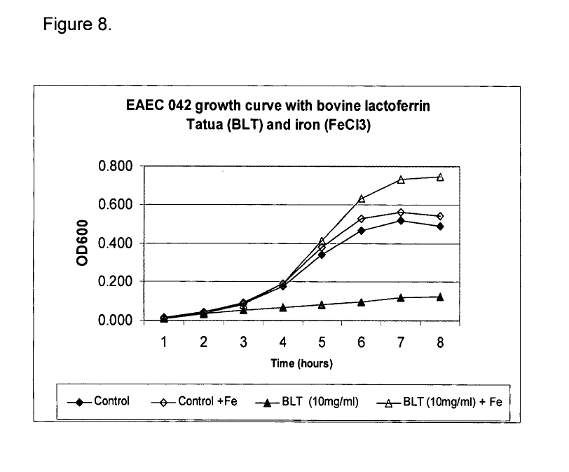

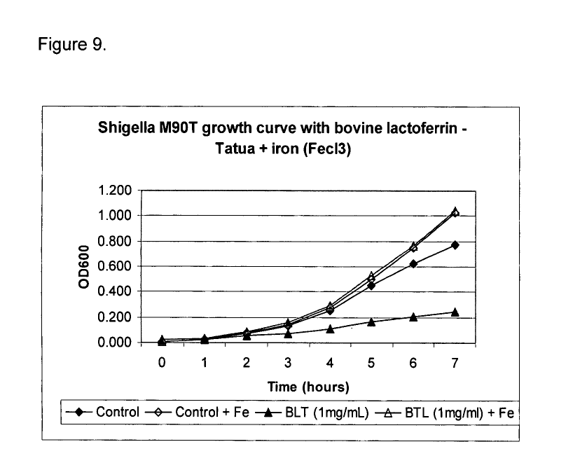

This example illustrates the effect of iron saturation on the growth inhibiting effect of bLF. To compare the iron saturation of each lactoferrin preparation, samples (10 mg/mL) were diluted in 0.1 M Tris, pH 8.5 and placed on a rotisserie for 2 hours. The iron saturation was judged by iron content determination by absorbance at 465 nm (Mazurier and Spik, 1980). The protein content was estimated by absorbance at 280 nm. The iron/protein ratios for the various lactoferrin preparations were related to growth. In this example, a saturating amount of iron reversed the growth-inhibiting effect of bLF on the bacteria (FIGS. 7-9).

### EXAMPLE 8

This example illustrates the effectiveness of bLF that has been isolated from whole milk and has a low somatic cell count versus bLF preparations that were isolated from powdered milk and do not have a low somatic cell count. Comparative studies were conducted using commercially available lactoferrin preparations, shown in Table 1.

TABLE 1

| Study | | | |
|---|---|---|---|
| code | Type | Trade name | Manufacture |
| bLF-Tatua | Bovine | Bovine lactoferrin | Tatua Nutritionals, Tatua |
| | | | Co-operative Dairy Co, |
| | | | Ltd, Morrinsville, New |
| | | | Zealand |
| bLF-BF20 | Bovine | Bioferrin ™ 2000 | Glanbia Nutritional, Inc, |
| | | | Monroe, WI |
| bLF-BF10 | Bovine | Bioferrin ™ 1000 | Glanbia Nutritional, Inc, |
| | | | Monroe, WI |
| bLF-FD | Bovine | LF-FD domestic | DMW international |
| | | lactoferrin | nutritional, Delhi, New |
| | | | York |
| bLF-NZMP | Bovine | Bovine lactoferrin | Fonterra Ltd, Auckland, |
| | | NZMP | New Zealand |
| bLF-Sigma | Bovine | Bovine lactoferrin | Sigma - Aldrich CO. St |
| | | | Louis, MO |

These lactoferrin preparations were approximately 10-20% iron saturated. The lactoferrin preparations were diluted in sample buffer (2-mercapthoethanol, sodium dodecyl sufate and 0.1% bromphenol blue) at a final concentration of 1 mg/mL. Samples were then resolved on 10% sodium dodecyl polyacrylamide gel electrophoresis (SDS-PAGE) and stained with coomassie blue or silver stain.

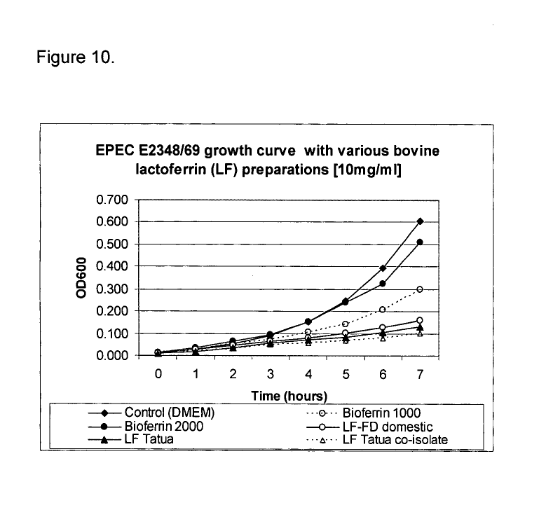

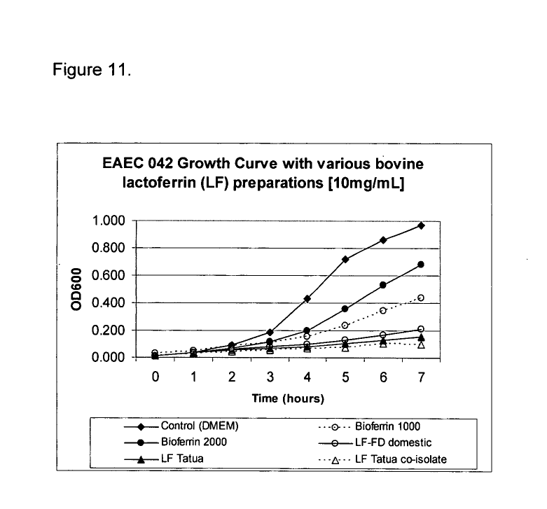

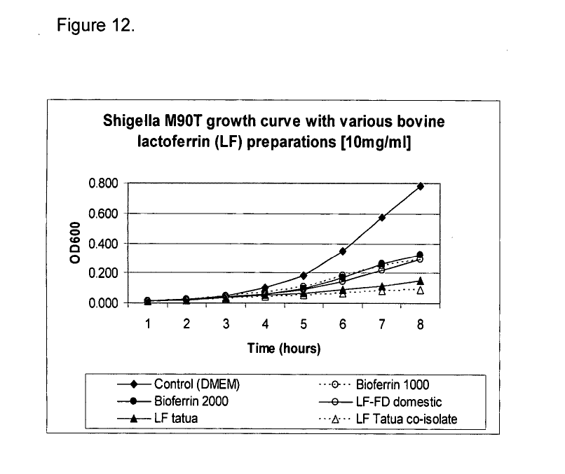

The various lactoferrin preparations differed in their inhibitory effect on EPEC, EAEC and Shigella growth (FIGS. 10-12). Each of the bLF preparations were effective at inhibiting the growth of EPEC, EAEC, and Shigella. In each experiment, however, LF-Tatua had the highest growth-inhibitory effect on EPEC, EAEC and Shigella. LF-Tatua was isolated from whole bovine milk and had a low somatic cell count.

This variability could possibly be explained by differences in their iron contents. The iron content of 5 lactoferrin preparations was evaluated: bLF-BF20, bLF-BF10, bLF-Tatua, and bLF-NZMP. For each lactoferrin preparation we estimated the iron content per mg protein by measuring their OD at 280 (for protein content) and 465 (for iron content). The ratio of iron/protein for each preparation was compared with its effect on EAEC, EPEC and Shigella growth at 6 hours. As expected, the preparation with the highest growth inhibition (lowed OD) had the lowest iron content. The linear regression analysis indicates an R-squared of 0.58 for EAEC, 0.55 for EPEC, and 0.81 for Shigella.

### EXAMPLE 9

This example illustrates the effect of bLF on EAEC attachment. The ability of bLF to block EAEC attachment was evaluated in a HEp-2-cell assay system. A sub-confluent layer of HEp-2 cells (approximately 5×10⁴ cells/well, in a 24-well plate) was infected with EAEC at bacteria-to-target ratio of approximately 100:1. Bacteria from overnight culture were grown in bLF for 4 hours, centrifuged, washed, and resuspended in adhesion media (DMEM, 100 mM HEPES, pH 7, with 1% D-mannose). This bacterial suspension, with and without bLF (10, 1, 0.1, 0.01 mg/mL), was incubated with the HEp-2 cells at 37° C. in 5% CO₂ for 4 hours. The monolayer was then washed vigorously to remove non-adherent bacteria. EAEC adherence was evaluated by microscopy and cfu. For microscopy, cells were fixed with 100% methanol and stained with crystal violet. For cfu evaluation, HEp-2 cells were treated with Triton X-100, serially diluted in phosphate-buffered saline (PBS) and cultured on MacConkey plates.

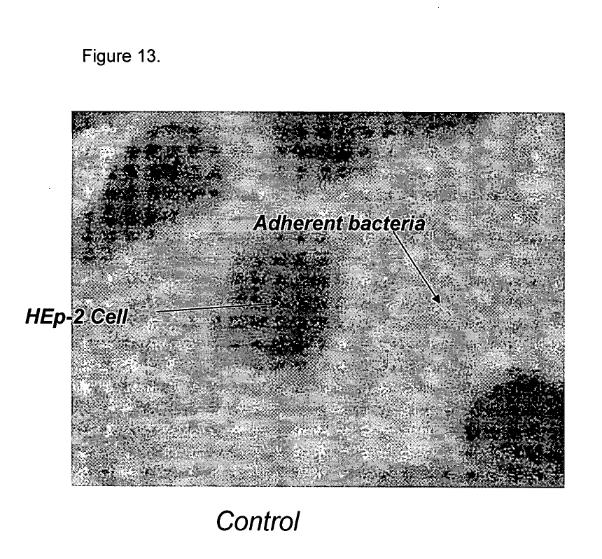

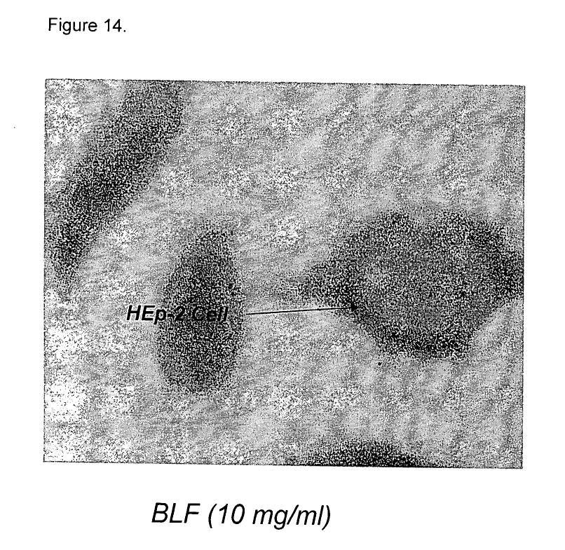

A blinded observer qualitatively evaluated the number of bacteria/HEp-2 cell (0-3+) by microscopy and the characteristic “stacked brick” aggregative pattern. In the absence of bLF there were 3+ bacteria/HEp-s cells with the characteristic aggregative pattern (FIG. 13). In the presence of bLF at 10 mg/mL (FIG. 14) and 1 mg/mL, there were no bacteria on the field. In the presence of bLF at 0.1 and 0.01 mg/mL, some bacteria diffusely adhered to HEp-2 cells, but without the typical aggregative pattern. In order to quantitatively characterize this effect, we measured the amount of bacteria attached to HEp-2 cells by cfus. bLF decreased adherence to HEp-2 cells by 82% and 73% for 10 and 1 mg/mL without iron saturation (p<0.05 for both).

### EXAMPLE 10

This example illustrates the effect of iron saturation on the adherence of EAEC to HEp-2 cells. At 10 mg/mL of bLF, the adherence of EAEC was almost completely inhibited. The inclusion of saturating amounts of iron did not affect the anti-adherence effect. At 1 mg/mL, the same result was observed.

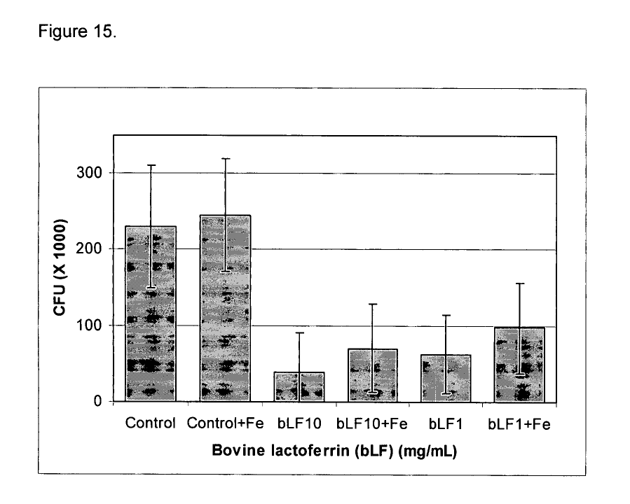

bLF saturated with iron decreased adherence to HEp-2 cells by 71% and 60% for 10 and 1 mg/mL, respectively (p<0.05 for both) (FIG. 15). The cfu in bLF preparations with or without iron saturation were not significantly different: 7.1±5.8×10⁴ cfu/mL (bLF 10 mg/mL with iron) vs. 4.0±5.1×10⁴ cfu/mL (bLF 10 mg/mL without iron) (p=0.386); and 9.8±5.9×10⁴ cfu/mL (bLF 1 mg/mL with iron) vs. 6.3±5.1×10⁴ cfu/mL (bLF 1 mg/mL without iron) (p=0.386). Thus, bLF decreases the adherence of EAEC to tissue culture cells under non-bacteriostatic conditions and this effect is not iron-dependent.

All references cited in this specification, including without limitation, all papers, publications, patents, patent applications, presentations, texts, reports, manuscripts, brochures, books, internet postings, journal articles, periodicals, and the like, are hereby incorporated by reference into this specification in their entireties. The discussion of the references herein is intended merely to summarize the assertions made by their authors and no admission is made that any reference constitutes prior art. Applicants reserve the right to challenge the accuracy and pertinence of the cited references.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged in whole or in part. Furthermore, those of ordinary skill in the art will appreciate that the foregoing description is by way of example only, and is not intended to limit the invention so further described in such appended claims. Therefore, the spirit and scope of the appended claims should not be limited to the description of the preferred versions contained therein.

What is claimed is:

1. A method for inhibiting the growth, in a subject, of a bacterial pathogen expressing a type III secretory system, the method comprising administering to the subject an effective amount of bovine lactoferrin.

2. The method according to claim 1, wherein the bacterial pathogen expressing a type III secretory system is selected from the group consisting of Salmonella, Shigella, Yersinia, Pseudomonas and Escherichia.

3. The method according to claim 1, wherein the bacterial pathogen expressing a type III secretory system is selected from the group consisting of enteropathogenic E. coli and enterohemorrhagic E. coli.

4. The method according to claim 3, wherein the enteropathogenic E. coli is E. coli E2348/69.

5. The method according to claim 3, wherein the enterohemorrhagic E. coli is selected. from the group consisting of STEC TWO 8023, STEC HW1 and STEC 306-7.

6. The method according to claim 1, wherein the bacterial pathogen expressing a type III secretory system is Shigella flexneri.

7. The method according to claim 6, wherein the Shigella flexneri is Shigella flexneri M90T.

8. The method according to claim 1, wherein the effective amount is in the range of about 0.001 mg to about 100 g daily.

9. The method according to claim 1, wherein the effective amount is in the range of about 0.1 mg to about 10 g daily.

10. The method according to claim 1, wherein the effective amount is in the range of about 10 mg to about 1,500 mg daily.

11. The method according to claim 1, wherein the bLF is provided in three daily doses.

12. The method according to claim 1, wherein the bovine lactoferrin has been isolated from whole milk

13. The method according to claim 1, wherein the bovine lactoferrin has a low somatic cell count.

14. The method according to claim 1, wherein the production of toxins by the bacterial pathogens is not increased.

15. The method according to claim 1, wherein the administration comprises spraying bovine lactoferrin onto the surface of a food product.

16. The method according to claim 1, wherein the administration comprises intermixing bovine lactoferrin with the ingredients of a food product.

17. The method according to claim 1, wherein the subject is in need of growth inhibition of a bacterial pathogen expressing a type III secretory system.

18. The method according to claim 1, wherein the subject is a child or infant.

19. The method according to claim 1, wherein the subject is an infant that is not fed human breast milk.

20. A method for preventing or treating, in a subject, an infection that is caused by a bacterial pathogen expressing a type III secretory system, the method comprising administering to the subject an effective amount of bovine lactoferrin.

21. The method according to claim 20, wherein the infection is selected from the group consisting of urinary tract infection, neonatal meningitis, peritonitis, shigellosis and gastrointestinal infections.

22. The method according to claim 20, wherein the bacterial pathogen expressing a type III secretory system is selected from the group consisting of Salmonella, Shigella, Yersinia, and Escherichia.

23. The method according to claim 20, wherein the bacterial pathogen expressing a type III secretory system is selected from the group consisting of enteropathogenic E. coli and enterohemorrhagic E. coli.

24. The method according to claim 20, wherein the subject is in need of prevention or treatment of an infection caused by a bacterial pathogen expressing a type III secretory system.

25. The method according to claim 20, wherein the subject is a child or infant.

26. The method according to claim 20, wherein the subject is an infant that is not fed human breast milk.

27. A method for inhibiting the adherence of bacterial pathogens expressing a type III secretory system to the intestinal wall of a subject, the method comprising administering to the subject an effective amount of bovine lactoferrin.

28. The method according to claim 27, wherein the bacterial pathogen expressing a type III secretory system is selected from the group consisting of Salmonella, Shigella, Yersinia, and Escherichia.

29. The method according to claim 27, wherein the bacterial pathogen expressing a type III secretory system is selected from the group consisting of enteropathogenic E. coli and enterohemorrhagic E. coli.

30. A method for causing the premature release of EspB in a bacterial pathogen expressing a type III secretory system, the method comprising contacting the bacterial pathogen with an effective amount of bovine lactoferrin.

31. The method according to claim 30, wherein the bacterial pathogen expressing a type III secretory system is selected from the group consisting of Salmonella, Shigella, Yersinia, and Escherichia.

32. The method according to claim 30, wherein the bacterial pathogen expressing a type III secretory system is selected from the group consisting of enteropathogenic E. coli and enterohemorrhagic E. coli.

33. A method for causing the degradation of EspB in a bacterial pathogen expressing a type III secretory system, the method comprising contacting the bacterial pathogen with an effective amount of bovine lactoferrin.

34. The method according to claim 33, wherein the bacterial pathogen expressing a type III secretory system is selected from the group consisting of Salmonella, Shigella, Yersinia, and Escherichia.

35. The method according to claim 33, wherein the bacterial pathogen expressing a type III secretory system is selected from the group consisting of enteropathogenic E. coli and enterohemorrhagic E. coli.

36. A method for inhibiting the growth of enteroaggregative E. coli in a subject, the method comprising administering to the subject an effective amount of bovine lactoferrin.

37. The method according to claim 36, wherein the bovine lactoferrin has been isolated from whole milk

38. The method according to claim 36, wherein the bovine lactoferrin has a low somatic cell count.

39. The method according to claim 36, wherein the enteroaggregative E. coli is E. coli O42.

40. The method according to claim 36, wherein the subject is in need of growth inhibition of enteroaggregative E. coli.

41. The method according to claim 36, wherein the subject is a child or infant.

42. The method according to claim 36, wherein the subject is an infant that is not fed human breast milk.

43. A method for preventing or treating an infection caused by enteroaggregative E. coli in a subject, the method comprising administering to the subject an effective amount of bovine lactoferrin.

44. The method according to claim 43, wherein the bovine lactoferrin has been isolated from whole milk

45. The method according to claim 43, wherein the bovine lactoferrin has a low somatic cell count.

46. The method according to claim 43, wherein the infection is selected from the group consisting of urinary tract infection, neonatal meningitis, peritonitis, shigellosis and gastrointestinal infections.

47. The method according to claim 43, wherein the enteroaggregative E. coli is E. coli O42.

48. The method according to claim 43, wherein the subject is in need of prevention or treatment of an infection caused by enteroaggregative E. coli.

49. The method according to claim 43, wherein the subject is a child or infant.

50. The method according to claim 43, wherein the subject is an infant that is not fed human breast milk.

51. A method for inhibiting the adherence of enteroaggregative E. coli to intestinal cells, the method comprising contacting the enteroaggregative E. coli with bovine lactoferrin.

52. The method according to claim 51, wherein the bovine lactoferrin has been isolated from whole milk.

53. The method according to claim 51, wherein the bovine lactoferrin has a low somatic cell count.

54. The method according to claim 51, wherein the enteroaggregative E. coli is E. coli O42.

55. An enteral formulation comprising bovine lactoferrin which has been isolated from whole milk and has a low somatic cell count.

56. The formulation according to claim 55, additionally comprising casein glycomacropeptide.

57. The formulation according to claim 55, additionally comprising at least one long-chain polyunsaturated fatty acid.

58. The formulation according to claim 57, wherein the long-chain polyunsaturated fatty acid is selected from the group consisting of DHA, ARA and combinations thereof.

59. The formulation according to claim 55, additionally comprising at least one probiotic.

60. The formulation according to claim 59, wherein the probiotic is selected from the group consisting of LGG, Bb-12 and combinations thereof.

61. The formulation according to claim 55, additionally comprising at least one prebiotic.

62. The formulation according to claim 61, wherein the prebiotic is selected from the group consisting of lactulose, galacto-oligosaccharide, fructo-oligosaccharide, isomalto-oligosaccharide, soybean oligosaccharides, lactosucrose, xylo-oligosacchairde and gentio-oligosaccharides.

63. The formulation according to claim 55, wherein the enteral formulation is an infant formula.
